(19) United States
(12) Patent Application Publication
(10) Pub. No.: US 2006/0263254 A1
(43) Pub. Date: Nov. 23, 2006

(54) Portable gas sensor and method for calibrating the same

(76) Inventor: Won Bae Lee, Daejeon (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/529,836

(22) PCT Filed: Jul. 29, 2003
(86) PCT No.: PCT/KR03/01518

§ 371 Date: Mar. 30, 2005

(30) Foreign Application Priority Data
Jul. 29, 2002 (KR) 10-2002-0044696

(51) Int. Cl. G01N 33/00

(52) U.S. Cl. 422/83; 422/98

(57) ABSTRACT

The present invention provides a portable gas sensor having a sensing material reacted with a target gas. The portable gas sensor includes a mode input unit for selecting an operation mode; a gas inhaling unit for accepting an external gas; a sensing unit for outputting a voltage value corresponding a varied resistance value of the sensing material reacted with the external gas; a first memory for storing a reference voltage value corresponding a resistance value of the sensing material reacted with a reference gas; a calibration control unit for storing a first voltage value corresponding to a resistance value of the sensing material reacted with a substitute reference gas in the first memory by substituting the reference voltage value; a target gas sensing control unit for storing a second voltage correspond to a resistance value of the sensing material reacted with the target gas into the second memory; a comparison and calculation unit for comparing and calculating the first voltage value and the second voltage value; and a display unit for displaying a concentration of the target gas calculated in the comparison and calculation unit.

### TECHNICAL FIELD

The present invention relates to a gas sensor; and, more particularly, to a portable gas sensor and a method for calibrating the same.

### BACKGROUND ART

As industrial developments have been accelerated ever since the Industrial Revolution, problems related to atmospheric pollution caused by using such hazardous gases as carbon monoxide (CO), hydrogen sulfide (H₂S), sulfur dioxide (SO₂) and nitrogen oxide (NOₓ) have been increasingly issued. Also, danger of gas explosion and gas poisoning has been alerted.

After the first report on a catalytic combustion-type sensor by Johnson, various types of the gas sensor have been developed. Particularly, a semiconductor-type gas sensor, first discovered by Seiyama and Taguch, detects the presence or absence of a specific gas and its concentration through estimation of changes in resistance by using sintered materials, e.g., tin dioxide (SnO₂), zinc oxide (ZnO), and indium trioxide (In₂O₃), which are metal-oxide semiconductor materials. The semiconductor-type gas sensor was first commercialized in 1968 by the company, Figaro, in Japan and has been used mainly in a gas leakage alarming system and a gas concentration measurement system.

In the course of developing various types of the gas sensor, they have been modified to meet the purpose of use and detection of a certain desired gas: through improvements on employed materials and applicable sensing apparatuses. As a result of these efforts, various types of the gas sensor have been commercialized to be used in industries, medical fields and daily lives. Particularly, wide distribution of liquefied natural gas (LNG) and liquefied petroleum gas (LPG) and public attention to socially issued problems of drunk driving has led the gas sensor to be further commercialized in different types such as a portable leaking gas sensor or a portable alcohol analyzer.

Semiconductor is classified into an n-type and a p-type depending on conductivity mechanisms. Tin dioxide (SnO₂), which is the most typical sensitizer, is one of the n-type semiconductors. The number of positive ions of Sn is quantitatively less than that of negative ions of O and thus, producing unpaired electrons which contribute to a degree of conductivity. This SnO₂ then adsorbs oxygen in atmosphere to balance the number of positive and negative ions. Because of anionic characteristics of the adsorbed oxygen, electrons contributing to the conductivity of the semiconductor become locally captured in a surface of the adsorbed oxygen. As a result of this capturing state, the electrons lose their conductivity.

If the SnO₂ with the adsorbed oxygen is exposed to a reduction gas, e.g., CO and ammonia (NH₃), the adsorbed oxygen react with such reduction gas and become desorbed from the SnO₂ as shown in the following chemical equation.
2CO+O₂→2CO₂ Eq. 1
As shown in the Eq. 1, the captured electrons become free and contribute to the conductivity. Therefore, a specific gas type to be detected determines a degree of the conductivity of a semiconductor sensor. The detection of changes in the conductivity degree provides information on the presence or absence of a specific desired gas and its concentration if presents. Such material as platinum (Pt), gold (Au) and silver (Ag) are also added to the sensitizer SnO₂ as a catalyst in order to increase a sensing ability.

For a conventional portable gas sensor, there exists a problem of frequent errors caused by a different operating mechanism from a gas alarm which is continuously operated for 24 hours a day and 365 days a year. That is, the portable alcohol analyzer or portable leaking gas sensor is operated only in need. However, this type of the gas sensor also reacts with undesired gases depending on external environmental factors, e.g., temperature, humidity, atmospheric pressure and subsequently changes a reference value of the gas sensor. As a result, measurements by the gas sensor become deviated in a greater extent.

FIG. 1 is a graph showing changes in resistance depending on a concentration of alcohol in a portable alcohol analyzer. Typically, the relationship between the resistance change and the alcohol concentration in the conventional semiconductor gas sensor is expressed algebraically. However, this algebraic relationship is expressed linearly in FIG. 1.

Assuming that the conventional semiconductor-type gas sensor follows characteristics of the curve I shown in FIG. 1, the resistance is calibrated to about 20 KΩ when about 80 PPM of alcohol gas is injected into the gas sensor. Based on this calibration, a correspondent gas concentration can be easily determined through interpolation with the known measured resistance value.

FIG. 2 is a circuit diagram showing equivalent circuits of a bulk-type semiconductor gas sensor. A resistance value of a sensing material is different from each sensor. As shown in the equivalent circuits in FIG. 2, although properties of a sensing material vary corresponding to external environmental factors such as temperature and humidity, output values of the gas sensor are set to be identical with use of a variable resistance VR during an initial fabrication of the above gas sensor. Once the resistance values are calibrated to be identical under a predetermined consistent gas concentration by using the variable resistance VR, a relative gas concentration can be easily determined. The reference notations ‘Vh’, ‘Rh’, ‘Rs’, ‘Vcc’ and ‘Vout’ express a heater voltage, a heater resistance, a signal resistance, a power voltage and an output voltage, respectively.

However, accuracy of the gas sensor decreases if characteristics of the gas sensor change from the curve I to the curve II or III as a resistance value of the sensing material changes by the external environmental factors. For example, if the characteristic of the gas sensor is changed from the curve I to the curve II, the resistance value of the gas sensor is about 10 KΩ with respect to about 80 PPM of the alcohol gas and a calibrated concentration of the alcohol gas is about 320 PPM. Meanwhile, if the characteristic of the gas sensor following the curve I changes to the curve III, the resistance value of the gas sensor is about 30 KΩ with respect to about 80 PPM of the alcohol gas and the alcohol gas concentration is expressed in about 20 PPM based on the reference calibration. That is, there exists a high risk of deviations in the detection result according to changes in characteristics of the gas sensor.

In that case, another calibration is required. However, it is difficult to obtain a conveniently applicable reference gas, and the calibration proceeds manually by a manufacturer since the calibration skill has not been developed yet.

### DISCLOSURE OF THE INVENTION

It is, therefore, an object of the present invention to provide a portable gas sensor allowing a convenient calibration by a user and a method for calibrating the same.

In accordance with an aspect of the present invention, there is provided a portable gas sensor having a sensing material reacting to a target gas, including: a mode input unit for selecting an operation mode; a gas injecting unit bringing in an external gas; a sensing unit outputting a voltage value corresponding to a changed resistance value of the sensing material due to a reaction between the sensing material and the injected external gas; a first memory storing a reference voltage value corresponding to a resistance value of the sensing material with respect to a reference gas; a calibration control unit for storing a first voltage value in the first memory by replacing the reference voltage value stored in the first memory through an operation of a calibration mode initiated in response to an input from the mode input unit, the first voltage value corresponding to the changed resistance value of the sensing material reacting with a substitutionary reference gas; a target gas sensing control unit for storing a second voltage value in a second memory by operating a target gas measurement operation mode initiated in response to an input of the mode input unit, the second voltage value corresponding to a changed resistance value of the sensing material reacting with the target gas transferred through the gas injecting unit; a comparison and calculation unit for comparing the first voltage value with the second voltage value and estimating the comparison value; and a display unit displaying a concentration of the target gas estimated from the comparison and calculation unit.

In accordance with another aspect of the present invention, there is also provided a method for calibrating a portable gas sensor having a sensing material reacting to a target gas, including the steps of: storing a reference voltage value corresponding to a resistance value of the sensing material with respect to a reference gas in a memory; inputting a calibration operation mode by maneuvering a key operation; measuring a fist voltage corresponding to a resistance value of the sensing material with respect to a substitutionary reference gas; and storing the first voltage value in the memory by replacing the stored reference voltage value.

In accordance with still another aspect of the present invention, there is also provided a computer readable recording medium storing instructions for implementing the method for calibrating the portable gas sensor having the sensing material reacting to the target gas, the computer readable recording medium including the instructions of: storing a reference voltage value corresponding to a resistance value of the sensing material with respect to a reference gas; inputting a calibration operation mode by maneuvering a key operation; measuring a fist voltage corresponding to a resistance value of the sensing material with respect to a substitutionary reference gas; and storing the first voltage value in the memory by replacing the stored reference voltage value.

### BRIEF DESCRIPTION OF THE DRAWINGS

Other objects and aspects of the invention will become apparent from the following description of the embodiments with reference to the accompanying drawings, in which:

FIG. 1 is a graph showing changes in resistance depending on a concentration of alcohol gas in a conventional portable alcohol analyzer;

FIG. 2 is a circuit diagram showing equivalent circuits of a conventional bulk-type semiconductor gas sensor;

FIG. 3 is a block diagram of a portable gas sensor in accordance with a preferred embodiment of the present invention;

FIG. 4 is a graph illustrating an example of a lookup table for a blood alcohol-concentration (BAC) in accordance with the preferred embodiment of the present invention; and

FIG. 5 is a flowchart showing a calibration operation procedure and a measurement operation procedure of the portable gas sensor in accordance with the preferred embodiment of the present invention.

### BEST MODE FOR CARRYING OUT THE INVENTION

Hereinafter, a preferred embodiment of a portable gas sensor allowing a convenient calibration by a user and a method for calibrating the same will be described in detail referring to the accompanying drawings.

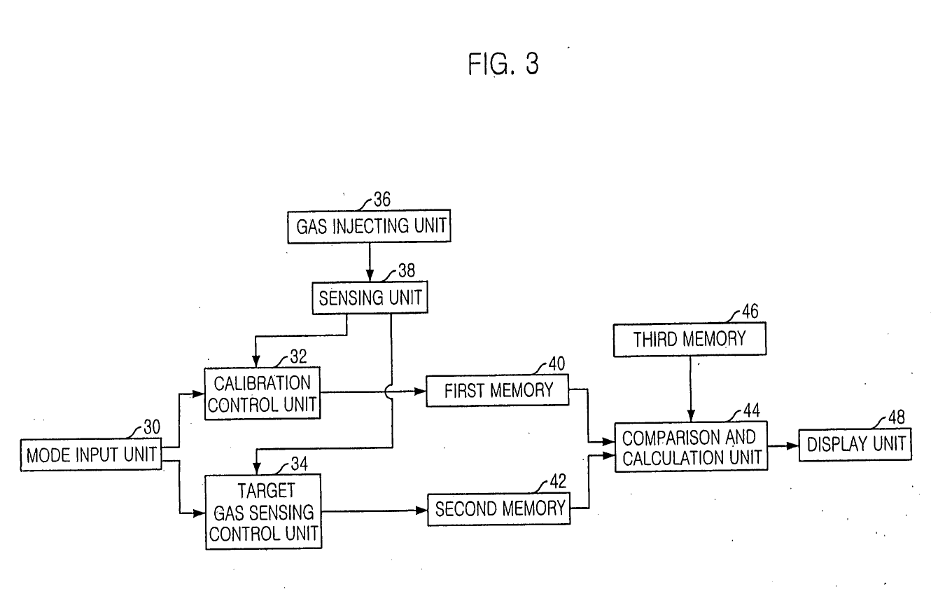

FIG. 3 is a block diagram showing the portable gas sensor in accordance with the preferred embodiment of the present invention.

Referring to FIG. 3, the portable gas sensor includes a mode input unit 30, a calibration control unit 32, a target gas sensing control unit 34, a gas injecting unit 36, a sensing unit 38, a first memory 40, a second memory 42, a comparison and calculation unit 44, a third memory 46 and a display unit 48. A user selects a calibration operation mode or a target gas measuring operation mode of the portable gas sensor through the mode input unit 30. A gas from an external source is injected into the gas injecting unit 36, and the injected gas reacts with a sensing material of the sensing unit 38. Because of the chemical reaction between the injected gas and the sensing material, a resistance value of the sensing material changes and a voltage value correspondent to the changed resistance value is stored into the first memory 40 or the second memory 42 depending on the input of the mode input unit 30.

In accordance with the preferred embodiment of the present invention, during an initial manufacturing procedure, the first memory 40 is stored with a reference voltage value corresponding to a resistance value of the sensing material with respect to a reference gas. Afterwards, once the calibration operation mode is operated, a voltage value outputted from the sensing unit 38 is stored as a substitutionary reference voltage value under a control of the calibration control unit 32. Also, the second memory 42 stores an outputted voltage value from the sensing unit 38 under a control of the target gas sensing control unit 34. That is, the voltage value stored into the first memory 40 changes its value only when the calibration operation is performed and is then stored in a reflected value of this resistance change. The voltage value stored into the second memory is stored in a different value as the target gas is measured.

The comparison and calculation unit 44 compares the voltage values stored in the first memory 40 and the second memory 42 with each other, and the comparison value is then calculated into a concentration of the target gas by using a lookup table stored in the third memory 46. The estimated concentration is seen in a liquid crystal display (LCD) through the display unit 48.

Hereinafter, the calibration operation and the target gas measuring operation according to the present invention will be explained in more detail.

First, a user selects the calibration operation mode through the mode input unit 30. Then, the sensing material of the sensing unit 38 is heated up to a predetermined temperature for a specific time for initialization. After the initialization state is reached through the heat treatment to the sensing material, the sensing unit 38 senses a change in the resistance of the sensing material due to a chemical reaction between the sensing material and a substitutionary reference gas transferred from the gas injecting unit 36. Then, under a control of the calibration control unit 32, a fist voltage value corresponding to the changed resistance value is stored into the first memory 40 by replacing the previously stored voltage value.

In addition to the calibration mode operation, a user can also select the target gas measuring operation mode through the mode input unit 30. Then, the sensing material of the sensing unit 38 is heated up to a predetermined temperature for a specific time for initialization. After the initialization state is reached, the sensing unit 38 senses a change in resistance of the sensing material due to a chemical reaction between the sensing material and a target gas transferred from the gas injecting unit 36. Then, under a control of the target gas sensing control unit 34, a second voltage value corresponding to the changed resistance value is stored into the second memory 42 by replacing the previously stored voltage value. The comparison and calculation unit 44 compares the first voltage value stored into the first memory 40 with the second voltage value stored into the second memory 42, and the comparison value is then calculated into a concentration of the target gas by using the lookup table stored into the third memory 46. This estimated concentration of the target gas is seen in the LCD through the display unit 48.

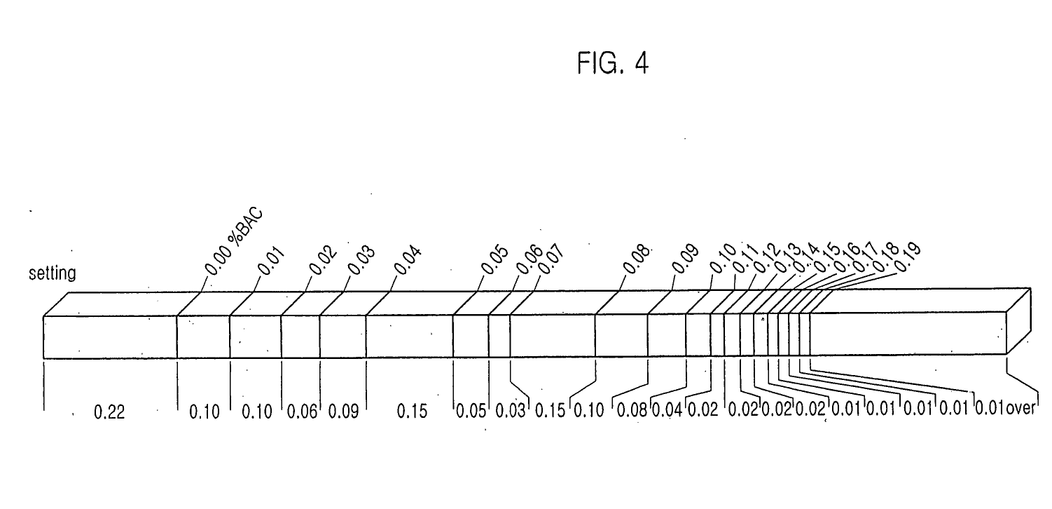

FIG. 4 is a graph showing an exemplary lookup table with respect to a blood alcohol concentration (BAC) in accordance with the preferred embodiment of the present invention. Values in a bottom portion of the lookup table express a voltage difference between a first voltage value and a second voltage value, while values in an upper portion of the lookup table express a BAC corresponding to the voltage difference.

Referring to FIG. 4, in case that the voltage difference between the first voltage value and the second voltage value is less than about 0.22 V, the BAC of about 0.00% is shown in the LCD through the display unit 48. This result is because even a breath without containing alcohol can have a voltage difference in about 0.22 V. For instance, if the voltage difference between the first voltage value and the second voltage value is about 0.42 V, the BAC of about 0.002% is shown in the LCD of the display unit 48. The values of the lookup table shown in FIG. 4 are experimental data and can be applied differently depending on each target gas type.

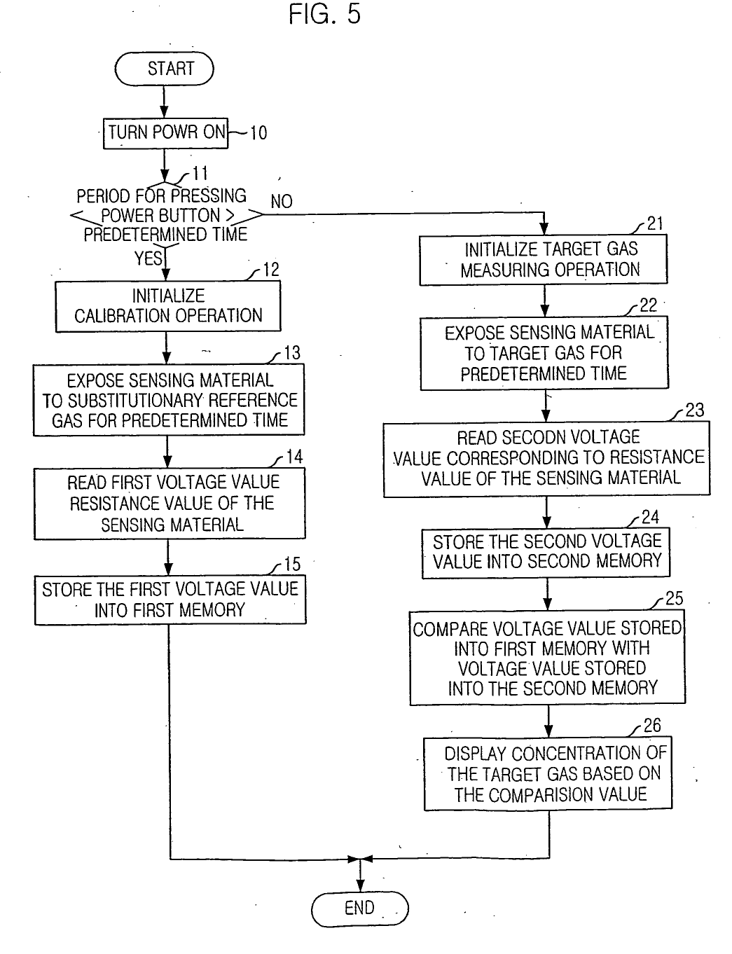

FIG. 5 is a flowchart showing a calibration operation procedure and a measurement operation procedure of the portable gas sensor in accordance with the preferred embodiment of the present invention.

More specifically, the calibration operation mode is initiated once a user who wants to use the calibration operation mode presses an externally disposed calibration operation mode button of the mode input unit 30 in the portable gas sensor. If the user wants to use a target gas concentration measuring operation mode, he/she presses the measurement operation mode button of the mode input unit 30 to initiate the measuring procedure. In the preferred embodiment of the present invention, the portable gas sensor does not have separate buttons for each operation mode but have one power button that differentiates the calibration operation mode and the measurement operation mode by how long the power button is pressed. That is, the calibration operation mode is selected by pressing the power button with above a predetermined time, and if otherwise, the target gas measurement operation mode is selected.

Referring to FIG. 5, the portable gas sensor is turned on by pressing the power button at Step 10. Then, at Step 11, it is determined whether the power button is pressed with above a predetermined time. If the power button is pressed for longer than the predetermined pressing time, the calibration operation mode is initialized at Step 12. Then, the sensing material is exposed to a substitutionary reference gas provided through the gas injecting unit 36 at Step 13. At this time, the substitutionary reference gas can be a human breath or air/atmosphere. The calibration control unit 32 recognizes the injected gas during the calibration operation mode as a reference gas, e.g. a human breath.

Next, at Step 14, the calibration control unit 32 reads a first voltage value corresponding to a changed resistance value of the sensing material from the sensing unit 38 by the injection of the substitutionary reference gas.

Thereafter, at Step 15, the calibration control unit 32 replaces the previously stored voltage value with the first voltage value corresponding to the changed resistance value of the sensing material and stores the first voltage in the first memory 40. Then, the process is terminated.

If the power button is pressed for less than the predetermined pressing time, the target gas measurement operation mode is initialized at Step 21. Then, the sensing material of the sensing unit 38 is exposed to the target gas provided by the gas injecting unit 36 for a predetermined time at Step 22.

Next, at Step 23, the target gas measurement control unit 34 reads a second voltage value corresponding to a resistance value of the sensing material after the exposure of the sensing material to the target gas.

Subsequent to Step 23, the second voltage value is stored into the second memory 42 under a control of the target gas sensing control unit 34 at Step 24. Then, the first voltage value and the second voltage value are compared with each other by the comparison and calculation unit 44 at Step 25. The resultant comparison value is substituted into the lookup table stored in the third memory 46 to estimate a concentration of the target gas corresponding to the comparison value. This estimated concentration is shown in the LCD of the display unit 4S, and the process is terminated.

Although the preferred embodiments of the invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

In the preferred embodiment of the present invention, a user oneself re-initialization function is established in a portable gas sensor such as a portable alcohol analyzer and a portable leaking gas sensor, and a user can conveniently maneuver the calibration operation mode by using a gas which can be easily obtained, e.g., a human breath or air, as a substitutionary reference gas. Typically, the semiconductor gas sensor has reactivity to various gases such like CH based gas, H₂O and CO. However, the reactivity of each semiconductor gas sensor is slightly different from each other depending on catalysis and temperature. This fact enables the semiconductor gas sensor to sense a particular gas. Based on this useful and unique characteristic of the semiconductor gas sensor, the inventive portable gas sensor is developed. That is, a breath exhaled into the semiconductor gas sensor shows a similar reaction pattern to an alcohol concentration of about 10 PPM even though there is a slight variation. For instance, since a typical human breath can be considered to be a substitutionary reference gas of about 10 PPM, this type of gas is referred as the substitutionary reference gas in the present invention. Based on this fact, this type of breath is first exhaled into the gas injecting unit of the portable gas sensor, and a new reference value is set by reading a resistance value of the sensing material of the sensing unit in the portable gas sensor reacting to the injected gas.

Meanwhile, in case of applying the preferred embodiment of the present invention to a leaking gas sensor for LPG or LNG, the gas sensor is required to be more sensitive compared to the alcohol analyzer. Therefore, it is preferable to take unpolluted air as a reference gas instead of using the human breath. Since the unpolluted air has a consistent composition ratio, it can be used as the relatively accurate reference gas.

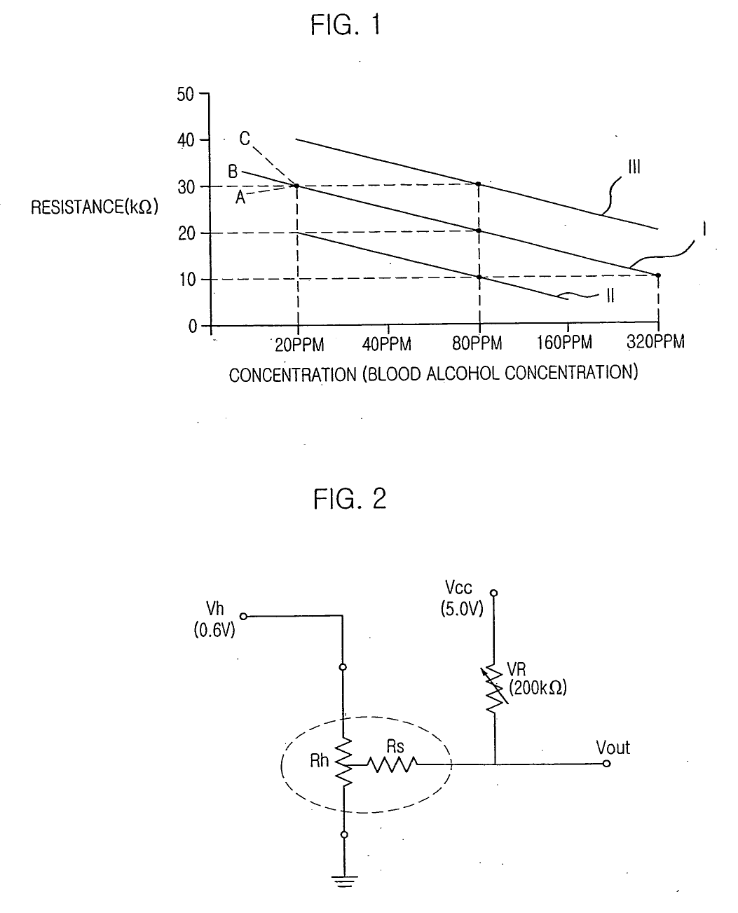

Nevertheless, if a slope of a characteristic curve shown in FIG. 1 loses its linearity at a low concentration, the curve I could have different resistance values like A, B and C. If the concentration is estimated in consideration of these deviated resistance values, it is possible to sense a gas sensitively even if the characteristic of the gas sensor is changed from the curve I to curve II or III.

Although the preferred embodiment of the present invention provides the exemplary portable alcohol analyzer and portable leaking gas sensor, it is still possible to apply the present invention to many other types of a portable gas sensor.

Also, although use of a human breath or air is described in the preferred embodiment of the present invention, any easily attainable gas having a consistent composition ratio can be used as the substitutionary reference gas for the calibration operation mode. Furthermore, a convenient maneuver of the calibration operation mode by a user provides an effect on enhancing accuracy of the gas sensor.

What is claimed is:

1. A portable gas sensor having a sensing material reacting to a target gas, comprising:
a mode input unit for selecting an operation mode;
a gas injecting unit bringing in an external gas;
a sensing unit outputting a voltage value corresponding to a changed resistance value of the sensing material due to a reaction between the sensing material and the injected external gas;
a first memory storing a reference voltage value corresponding to a resistance value of the sensing material with respect to a reference gas;
a calibration control unit for storing a first voltage value in the first memory by replacing the reference voltage value stored in the first memory through an operation of a calibration mode initiated in response to an input from the mode input unit, the first voltage value corresponding to the changed resistance value of the sensing material reacting with a substitutionary reference gas;
a target gas sensing control unit for storing a second voltage value in a second memory by operating a target gas measurement operation mode initiated in response to an input of the mode input unit, the second voltage value corresponding to a changed resistance value of the sensing material reacting with the target gas transferred through the gas injecting unit;
a comparison and calculation unit for comparing the first voltage value with the second voltage value and estimating the comparison value; and
a display unit displaying a concentration of the target gas estimated from the comparison and calculation unit.

2. The portable gas sensor as recited in claim 1, further comprising a third memory for storing a lookup table for determining a concentration of the target gas depending on a value difference between the first voltage value and the second voltage value.

3. The portable gas sensor as recited in claim 1, wherein the substitutionary reference gas is a human breath or air/atmosphere.

4. The portable gas sensor as recited in claim 1, wherein the mode input unit selects an operation mode by using a time difference in pressing a power button.

5. A method for calibrating a portable gas sensor having a sensing material reacting to a target gas, comprising the steps of:
storing a reference voltage value corresponding to a resistance value of the sensing material with respect to a reference gas in a memory;
inputting a calibration operation mode by maneuvering a key operation;
measuring a fist voltage corresponding to a resistance value of the sensing material with respect to a substitutionary reference gas; and
storing the first voltage value in the memory by replacing the stored reference voltage value.

6. The method as recited in claim 5, wherein the substitutionary reference gas is a human breath or air/atmosphere.

7. A computer readable recording medium storing instructions for implementing the method for calibrating the portable gas sensor having the sensing material reacting to the target gas, the computer readable recording medium comprising the instructions of:
storing a reference voltage value corresponding to a resistance value of the sensing material with respect to a reference gas;
inputting a calibration operation mode by maneuvering a key operation;
measuring a fist voltage corresponding to a resistance value of the sensing material with respect to a substitutionary reference gas; and
storing the first voltage value in the memory by replacing the stored reference voltage value.
